(19) United States
(12) Patent Application Publication
(10) Pub. No.: US 2008/0065136 A1
(43) Pub. Date: Mar. 13, 2008

(54) Distender device and method for treatment of obesity and metabolic and other diseases

(76) Inventor: Andrew Young, Rancho Santa Fe, CA (US)

Correspondence Address: DUANE MORRIS LLP, 101 WEST BROADWAY, SUITE 900, SAN DIEGO, CA, 92101-8285, US

(21) Appl. No.: 11/897,499

(22) Filed: Aug. 30, 2007

Related U.S. Application Data
(60) Provisional application No. 60/841,093, filed on Aug. 30, 2006.

(51) Int. Cl. A61M 29/00; A61F 2/04

(52) U.S. Cl. 606/191; 623/23.65

(57) ABSTRACT

A gastrointestinal implant device is positioned in a patient's small intestine or rectum and produces an outward force that itself produces a distension signal which is a therapeutically useful neural or humoral signal that evokes satiogenic or weight loss effects by itself. The device may advantageously be placed in the duodenum adjacent the pylorus or in the jejunum, ileum or rectum. The distension signals may amplify chemosensory or mechanosensory signals such as enteroendocrine secretions within the patient. The device may be a mesh and include a low material density that allows for unrestricted chyme absorption within the small intestine and unrestricted chyme flow through the gastrointestinal system. A method includes inserting the device into the patient then either retrieving the device after treatment is complete or allowing a device formed of a biodegradable material to degrade in time after treatment is complete.

### RELATED APPLICATION

This application is related to and claims priority of U.S. provisional application 60/841,093 filed Aug. 30, 2006, the contents of which are hereby incorporated by reference as if set forth in their entirety.

### FIELD OF THE INVENTION

The invention relates to a distender device and method for treating obesity and other metabolic diseases in a human or other animal.

### BACKGROUND

According to the World Health Organization (WHO), obesity has reached epidemic proportions globally—with more than 1 billion adults overweight, at least 300 million of them clinically obese—and is a major contributor to the global burden of chronic disease and disability. Non-fatal, but debilitating health problems associated with obesity include respiratory difficulties, chronic musculoskeletal problems, skin problems, and infertility. Overweight and obesity lead to adverse metabolic effects on body fat, cholesterol, triglycerides and insulin resistance and pose a major risk for chronic diseases. Life-threatening problems fall into several main areas: cardiovascular disease problems, including coronary artery disease, hypertension and stroke; conditions associated with insulin resistance including type 2 diabetes; certain forms of cancers, especially the hormonally related and large-bowel cancers; and gallbladder disease.

The likelihood of developing type 2 diabetes and hypertension rises steeply with increasing body fatness. Confined to older adults for most of the 20th century, this disease now affects obese children even before puberty. Approximately 85% of people with diabetes are type 2, and of these, 90% are obese or overweight and this is increasingly becoming a developing world problem.

Often coexisting in developing countries with under-nutrition, obesity is a complex condition, with serious social and psychological dimensions, affecting virtually all ages and socioeconomic groups. Increased consumption of more energy-dense, nutrient-poor foods with high levels of sugar and saturated fats, combined with reduced physical activity, have led to obesity rates that have risen three-fold or more since 1980 in some areas of North America, the United Kingdom, Eastern Europe, the Middle East, the Pacific Islands, Australasia and China. Health consequences range from increased risk of premature death to serious chronic conditions that reduce the overall quality of life.

The prevalence of overweight and obesity is commonly assessed by using body mass index (BMI), defined as the weight in kilograms divided by the square of the height in meters (kg/m²). A BMI over 25 is considered overweight, and a BMI of over 30 is considered obese. These markers provide common benchmarks for assessment, but the risks of disease in all populations can increase progressively from lower BMI levels. According to WHO, adult mean BMI levels of 22-23 are found in Africa and Asia, while levels of 25-27 are prevalent across North America, Europe, and in some Latin American, North African and Pacific Island countries. BMI increases among middle-aged elderly people, who are at the greatest risk of health complications.

The distribution of BMI is shifting upwards in many populations. Recent studies have shown that people who were undernourished in early life and then become obese in adulthood, tend to develop conditions such as high body fat, heart disease and diabetes at an earlier age and in more severe form than those who were never undernourished. Raised BMI also increases the risks of cancer of the breast, colon, prostrate, endometrium, kidney and gallbladder. Chronic overweight and obesity contribute significantly to osteoarthritis, a major cause of disability in adults. Although obesity should be considered a disease in its own right, it is also one of the key risk factors for other chronic diseases together with smoking, high body fat and high blood cholesterol. In the analyses carried out for World Health Report 2002, approximately 58% of diabetes and 21% of ischemic heart disease and 8-42% of certain cancers globally were attributable to a BMI above 21.

Of special concern is the increasing incidence of child obesity. Childhood obesity is already epidemic in some areas and on the rise in others. An estimated 22 million children under five years old are estimated to be overweight worldwide. According to the US Surgeon General, in the USA the number of overweight children has doubled and the number of overweight adolescents has trebled since 1980. The prevalence of obese children aged 6-to-11 years has more than doubled since the 1960s. Obesity prevalence in youths aged 12-17 has increased dramatically from 5% to 13% in boys and from 5% to 9% in girls between 1966-70 and 1988-91 in the USA.

Obesity is the second leading cause of preventable death in the United States. Approximately 127 million adults in the U.S. are overweight, 60 million obese, and 9 million severely obese. Obesity and diabetes currently account for about 280,000 early deaths per year in the U.S., comparable to smoking. The rate of increase of metabolic diseases is sufficiently high to be regarded by the World Health Organization as the first non-infectious epidemic. Obesity carries with it several other comorbidities that conspire to elevate the mortality rate 50-100%, the increased risk being predominantly cardiovascular. The major obesity-associated comorbidities include diabetes mellitus (type 2), hypertension, dyslipidemia (hypercholesterolemia and low HDL). The economic cost of obesity to the US in 1995 was $99.2 billion, represented by direct costs of $51.6 billion and indirect costs of $47.6 billion. The estimated total economic cost of obesity in the United States was about $117 billion in 2000. WHO reports that obesity accounts for 2-6% of total health care costs in several developed countries.

Weight loss can thus be a life saving measure and is the objective of many health care therapies. Weight loss as low as 2-10 percent can greatly improve body fat, blood sugar, and cholesterol and decrease need for medication.

Diet, exercise and lifestyle recommendations have proven to be mostly ineffective in adequately preventing or treating the progression of obesity. Dietary therapy with or without accompanying behavioral therapy may be effective initially, but long-term follow-up shows regain of the weight that was lost in most cases. It has been reported that 98% of those who achieve weight loss by diet have regained it within 5 years.

The following includes information that may be useful in understanding the present invention. It is not an admission that any of the information provided herein is prior art, or relevant, to the presently described or claimed invention, or that any publication or document that is specifically or implicitly referenced, is prior art.

Of approved pharmacotherapies, only 2 are known to currently marketed-orlistat and sibutramine. A further anti-obesity therapy, rimonobant, has recently gained marketing approval in some countries. The utility of current pharmacotherapies has been limited by modest efficacy and the high incidence of side effects. Negative consequences can be devastating to patients and to manufacturers. At least seven drugs are known to have been withdrawn from the market due to toxicity or other failure. The magnitude of the latent demand for effective and safe therapies is however reflected in annual expenditures of $33B in the U.S. for over-the-counter therapies, nutritional therapies, and “fringe” medicines, most having trivial or unproven benefit.

Surgery is one exemplary therapy for addressing obesity and the comorbidities of obesity, including diabetes, hypertension, dyslipidemia, gastric reflux disease, and arthritis. Of about 20 different surgeries that have been attempted for the treatment of morbid obesity, the Applicant is aware of about 6 that remain, including the Roux-en-Y gastric bypass (RYGBS), with biliopancreatic diversion. Vertical banded gastroplasty restricts the size of the stomach using a stapling technique. Laparoscopic versions of surgical procedures are also performed.

The Roux-en-Y procedure has enjoyed a level of success in terms of weight loss and other metabolic benefits. In 2003, approximately 140,000 such procedures were reportedly performed within the U.S., up from about 10,000 in 1998. It is unlikely, due to the rate at which new surgeons can be trained and operating rooms made available,.that this number could extend beyond approximately 200,000 per year in the near future. At the same time, the number of patients eligible for such surgery in the U.S. is at least 12 million, and depending upon criteria established largely by insurers, may be as high as 23 million. Alternatively stated, there appears to be a shortage in the number of surgeons available to perform surgeries on the patients eligible for such surgeries in the U.S. Moreover, bariatric surgery is expensive (over $30,000), mortality is 0.5-1.5%, and over 10% of cases develop complications requiring surgical correction.

For these and other reasons, there is an acute need for less expensive interventions, with durable effect, that can be performed faster and with less risk, but which can mimic certain benefits of bariatric surgery.

Several devices have been developed to emulate the processes which many have interpreted to underly the efficacy of bariatric surgery. Such processes have historically included gastric factors. Gastric factors include (1) reduced gastric size, (2) increased sensations of gastric distension, and (3) reduced production of the orexigenic hormone, ghrelin. Gastric banding is one technique for treating obesity that involves placing an externally adjustable gastric band around the outside of the stomach. The stomach is not entered by the gastric banding apparatus.

Devices and procedures that aim to reduce gastric size include the Sapala-Wood Micropouch procedure such as in U.S. Pat. No. 6,758,219. Another surgical procedure is a constrictive coating applied to the outside of the stomach such as in U.S. Pat. No. 6,572,627 to Gabbay. One device is a tool to enable vertical band gastroplasty, a size-restricting procedure as in U.S. Patent App. 2004/0097989A1 to Trigueros. Some devices aim to bypass the accommodating volume and digestive environment of the stomach by the insertion of a gastric sleeve such as U.S. Patent App. 2004/0039452A1 of Bessler.

Devices and procedures which aim to restrict food influx into the stomach include previously described banding devices, such as an adjustable gastroplasty ring as in see U.S. Patent App. Publication 2004/0049209A1 to Benchetrit; banding procedures [see U.S. Patent App. 2004/0097989A1 (Trigeros)]; an adjustable banding device [see U.S. Pat. No. 4,592,339 (Kuzmac et al.)]; and, an implanted restrictor at the gastro-esophageal junction as in WO 03/086246A1 to Stack et al., WO 2004/064680A1 to Stack et al. and the positioning tool in WO 2004/064685A1.

Other devices aim to create an artificial distension signal in the stomach only, either by occupying space, such as with balloons as in WO 00235980A3 and WO 04019765A2. Other intragastric expanders are described in U.S. Pat. No. 6,675,809 to Stack et al. and U.S. Pat. No. 5,868,141 to Ellias.

Other approaches aim to moderate the rate of stomach emptying by local treatment of the pylorus, e.g. with pharmacologic agents as described in U.S. Patent App. publication 2004/0089313A1 or with electro stimulation [see U.S. Patent App. publication 2004/0015201A1].

Several approaches apply an impermeable barrier between the chyme, undigested food, and the absorptive intestinal wall, for varying lengths of the intestine. In one application, the barrier is applied as a liquid, or as a film bonded to the gut as in U.S. Pat. No. 4,315,509 and U.S. Patent App. 2003/0191476A1. Impermeable sleeves of various configurations have been described in U.S. Pat. No. 4,501,264, U.S. Pat. No. 5,306,300 and U.S. Pat. No. 5,820,584, WO 03/094785A1, and WO 04/049982A2, for example. The sleeves principally vary in their point of origination, some anchored within the stomach, and some distal. One sleeve device provided in WO 03/094785A1 is anchored just below the esophageal sphincter so the sleeve isolates the stomach as well as continuing as a barrier to absorption within the proximal small bowel. Another device provided in U.S. Pat. No. 5,820,584 is based within the pylorus, with a tubular duodenal extension to delay intermixing of digestive enzymes with food exiting the stomach.

A flexible tubular screen that also aims to maintain separation between food and digestive juices [U.S. Pat. No. 5,306,300] has also been designed with a ring that is self-anchoring within the antrum, and a “brush-like” distal end that is subject to normal peristaltic forces to keep it extended within the gut. This device claims advantages over the devices of Smit, U.S. Pat. No. 4,315,509 and Rockey, U.S. Pat. No. 4,501,264. The sleeve of Rockey generally isolates any viscera from its detrimental contents, but in the context of obesity, was described only as being placed within the stomach to limit digestive processes therein. The bariatric sleeve of Levine et al. in WO 04/049982A2 and U.S. Patent App. 2004/0107004, is described as anchored in the stomach, with the barrier extending beyond the ligament of Treitz [U.S. Patent App. 2005/0080395]. Anchors have also been designed that sit just distal to the pylorus, within the duodenum [U.S. Patent App. 2005/0125020A1]. The intent and effect of each of these devices is to separate food from the absorptive surfaces of the gut. A further development in the form of an enzyme sleeve, aims to delay digestion by shunting digestive enzymes from the exocrine pancreas to distal intestinal sites as in U.S. Patent App. publication 2004/0249362A1 to Levine et al.

The working principles of all the above devices are essentially limited to: (1) restricting meal capacity and/or flow, and/or (2) applying a barrier to digestion and/or absorption.

Currently marketed devices for treating obesity include the aforementioned banding devices which have lesser efficacy in the treatment of morbid obesity than does Roux-en-Y gastric bypass surgery, and typically result in loss of about 50% of excess body weight. Nonetheless, sales of such devices are high, indicating a need for such devices despite their requiring invasive surgery for placement, an excess weight loss of only 38-45%, the need for periodic adjustment of the band, and complications in many patients.

The gut is functionally divisible into three general parts. Two of these parts, the stomach and the colon, exhibit high mechanical compliance in that they are able to accommodate large relative changes in volume with comparatively small changes in intraluminal pressure. These high-compliance segments are identified as having a storage role. For example, the stomach, in addition to its role in the liquifaction and initial digestion of chyme, is a repository capable of holding more food than the organism immediately needs and releasing it at a rate commensurate with digestive and absorptive capacity, into the period when the next uncertain meal might occur. Similarly, the colonic store can hold fecal material until most water, energy and other useful constituents have been scavenged, and until it is safe and opportune to eject the remainder. In between these two gut divisions resides a less compliant segment, the small intestine, which is specialized for digestion and absorption but has been the subject of fewer studies with respect to approaches for treating obesity.

Prior approaches in creating artificial signals of hollow organ distension for the treatment of obesity and other metabolic diseases have focused upon the stomach but not other portions of the gut. Such approaches, described above, include balloons, distenders, and other space-occupying devices. Additionally, devices that stimulate the vagus nerve at the stomach may function by creating neural traffic simulating that invoked by distension, and thereby also constitutes an artificial distension signal.

Flow of chyme through the small intestine is rigidly controlled in response to several indicators. Entry from the stomach into the small bowel is controlled via several feedbacks. For example, the entry of acid from the stomach into the duodenum does not exceed the rate at which bicarbonate secreted from the exocrine pancreas can neutralize it. Control is mediated via the duodenal hormone, secretin, which slows gastric emptying, slows gastric acid secretion, and stimulates bicarbonate secretion. Similarly, fat enters the duodenum at a rate no faster than that at which it can be emulsified by bile salts. These are synthesized in the liver, and expelled from the gallbladder to assist in lipase-mediated digestion of fats, all being processes stimulated by the duodenal/jejunal hormone cholecystokinin (CCK). CCK also slows gastric emptying as a mode of regulating fat entry into the small bowel. Similar controls are exerted by nutrients that might reach the distal small bowel without absorption. Such controls constituting the ileal brake are mediated via such hormones as GLP-1, PYY and oxyntomodulin, secreted from L-cells, nutrient-sensitive enteroendocrine cells in the mucosa. The tissue content of L-cell hormones increases with more distal passage down the gut, the effect being that the further down the digestive tract nutrient passes, the more vigorous nutrient-stimulated feedback control via enteroendocrine hormones becomes.

Additionally, the small bowel especially appears to exhibit autoregulation of its diameter in that distension of local parts is opposed via a localized contractile response. It is possible that this response is mediated via local enteric nervous system reflexes, and locally released mediators that could additionally have a systemic effect.

Beyond the stomach, scant attention has been paid to the role of mechanical signals, such as distension, localized pressure, and other hydraulic cues in the regulation of ingestion, gastric emptying, gut hormone secretion, and other metabolic controls.

In view of the widespread occurrence of obesity and related comorbities and the various attempts to treat the same, it would be useful to provide a less invasive method, device and system that both addresses the above-stated shortcomings and focuses on portions of the gut other than the stomach.

### SUMMARY OF THE INVENTION

To address the above and other needs and in view of its purposes, the present invention identifies segments of the gut other than the stomach, and in particular the small intestine, as important sources of signals of fullness, the generation of which will drive responses that are therapeutic in treating obesity and other metabolic diseases.

In one aspect, the invention provides a method for treating obesity/lowering body weight, reducing adiposity or reducing ingestion in a patient. The method comprises inserting at least one device capable of exerting an outward force in the patient's small intestine, the device allowing substantially unrestricted chyme absorption within the small intestine and substantially unrestricted chyme flow throughout the small intestine. The method further includes generating a small intestinal distension signal by exertion of the outward force by the device only, the outward force causing the small intestinal distension signal to thereby evoke weight loss or satiogenic effects by itself. The distension signals may be transmitted via neural pathways, humoral pathways, or both. In another aspect, the small intestinal distension signal may also amplify or augment one or more chemosensory signals within the patient.

According to another aspect, a method for treating obesity and/or lowering of weight or adiposity in a human provides inserting a device capable of imparting an expansile force in a human's small intestine, generating a small intestinal distension signal by expanding the device to provide a mechanical force sufficient to induce therapeutically useful signals that are at least one of neural signals and humoral signals that evoke satiogenic or weight loss effects by themselves while allowing for unrestricted chyme absorption within the small intestinal, unrestricted chyme flow through the small intestinal and unmodified further digestive functions. The small intestinal distension signal may also augment meal-related signals within the patient, wherein the meal-related signals are either generated responsive to a meal or are artificially generated responsive to a pharmaceutical or other therapy.

According to another aspect, a method for treating obesity and/or lowering of weight or adiposity or food intake in a patient, or alleviating the comorbities associated therewith, comprises inserting at least one device capable of exerting an outward force in the patient's rectum, generating a rectal distension signal by exertion of the outward force by the device only, the outward force causing the rectal distension signal to thereby evoke weight loss or satiogenic effects by itself; and the rectal distension signal amplifying one or more chemosensory signals within the patient.

According to another aspect, an insertable weight loss apparatus for a patient comprising a gastrointestinal implant is provided. The device consists of a discrete tubular-shaped device adapted for deployment in the patient's small intestine and for exerting physical pressure radially outward sufficient to evoke a therapeutically useful distension signal of satiety while allowing substantially unrestricted chyme flow through the small intestine and substantially unrestricted chyme absorption in the small intestine.

### BRIEF DESCRIPTION OF THE DRAWING

The present invention is best understood from the following detailed description when read in conjunction with the accompanying drawing. It is emphasized that, according to common practice, the various features of the drawing are not necessarily to scale. On the contrary, the dimensions of the various features are arbitrarily expanded or reduced for clarity. Like numerals denote like features throughout the specification and drawing.

FIG. 1 is flow chart that illustrates an exemplary method of the present invention;

FIG. 2 is a perspective view of one exemplary embodiment of the distension device of the present invention;

FIGS. 3A-3I are perspective views of other exemplary embodiments of the distension device of the present invention; and

FIG. 4 is a cross-sectional view showing an exemplary distension device situated within a patient's small intestine.

### DETAILED DESCRIPTION

The present invention provides many attributes and embodiments including, but not limited to, those set forth in this section which is not intended to be all-inclusive.

As used herein, “patient” refers to any animal classified as a mammal, including humans, domestic and farm animals, and zoo, sports, or pet animals, such as dogs, horses, cats, sheep, pigs, cows, etc. The preferred mammal herein is a human. As used herein, “preventing” means preventing in whole or in part, or ameliorating or controlling. As used herein, the term “treating” refers to both therapeutic treatment and prophylactic or preventative measures. Those in need of treatment include those already with the disorder as well as those prone to having the disorder or diagnosed with the disorder or those in which the disorder is to be prevented. “Treatments” include pharmacotherapy, nutritional treatments, other devices, surgery and other interventions, and combinations of these.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicant reserves the right to physically incorporate into this specification any and all materials and information from any such patents, publications, scientific articles, web sites, electronically available information, and other referenced materials or documents.

Applicant has discovered that volume/flow disturbances within the otherwise well-regulated small bowel are more evocative of therapeutically useful corrective responses than are disturbances in the more compliant gastric/colonic compartments.

A frequent unifying effect of chemosensory and mechanosensory feedback controls from the small bowel is a reduction in food intake, a slowing of gastric emptying, and a general reduction in digestive secretions. The terms “chemosensory” and “mechanosensory” apply to physiologic systems that respectively sense the chemical attributes of the contents of the gut, and its physical state. Signals impinging upon chemosensory systems include nutrients such as energy sources, material sources, vitamins, etc. as well as non-nutrient cues such as acidity, salinity, osmolality, bile salts, etc. Many chemosensory, and perhaps some mechanosensory, systems respond by secreting peptide hormones. Others respond by transmitting nerve impulses, locally or centrally. “Humoral” signals are blood-borne signals that include peptide hormones such as those secreted from gut enteroendocrine cells, but also non-peptide hormones. For example, a blood-borne humoral signal may be generated by enteroendocrine cells in response to a chemosensory stimulus within the gut lumen. Therapeutic approaches have utilized such humoral signals to benefit metabolic diseases. These include pharmaceutical approaches, wherein the signalling molecules or mimics thereof are administered, or where agents that enhance such signals, e.g., degrading enzyme inhibitors, are administered.

Electrophysiologic evidence for the existence of stretch receptors in the muscular layers of the duodenum was reported by Cottrell & Iggo in Cottrell, D. F. and Iggo, A. (1984), Tension receptors with vagal afferent fibres in the proximal duodenum and pyloric sphincter of sheep, J Physiol 354, 457-75. Yet data attesting the effects of duodenal distension on key metabolic responses is either absent or indirect. Recordings of the ventromedial hypothalamus (VMH) and lateral hypothalamus (LH), centers implicated in metabolic control, suggest an importance of duodenal mechanoreception. While gastric distension evoked mixed changes in the VMH and LH, recordings with duodenal distension universally decreased firing rate of LH units and increased firing rate of VMH units as reported in Maddison, S. and Horrell, R. I. (1979), Hypothalamic unit responses to alimentary perfusions in the anesthetised rat, Brain Res Bull 4 (2):259-66. 0361-9230. Moreover, VMH firing rate was increased by glucose; that is, distension and nutrient evoked VMH responses in the same direction.

In one known clinical study of healthy volunteers, a duodenal bag was inflated to volumes up to 48 mL while various distension/satiety-related perceptions were collected in Lingenfelser, T., Sun, W., Hebbard, G. S., Dent, J. and Horowitz, M. (1999), Effects of duodenal distension on antropyloroduodenal pressures and perception are modified by hyperglycemia, Am J Physiol 276, G711 -G718. Perceptions of duodenal pressure, duodenal fullness, satiety, and nausea were monotonically related to inflation volume, and were amplified by concomitant hyperglycemia. The inflated bag was a clinical test trial, however, and did not allow for chyme to pass through the subject's small intestine or any other portion of the gut, much less be absorbed by the walls of the gut.

These findings support Applicant's current finding that distension signals originating from small bowel evoke therapeutically useful responses.

Devices directed to applying an expansile force to the duodenum include an electrode array described in PCT application WO2005041749 wherein an intraluminal mesh applies sufficient pressure to ensure good electrical contact, and in US Patent Application publication 2005/0125020A1 which teaches a spring shaped as an annular wave designed to apply sufficient outward radial force to engage the barbs of an anchor of an impermeable bariatric sleeve, preventing chyme absorption. These references do not teach or suggest invoking a therapeutically useful distension signal from the applied outward force itself, however.

The present invention provides a device and method that imparts an expansile or other outward physical/mechanical force upon the rectum or the intestinal wall, typically the small intestine/duodenum thereby distending the anatomy, without restricting chyme flow, or modifying other digestive or absorptive processes. According to various exemplary embodiments, the device configuration may be an annular wave radial spring or it may take the shape of various coiled springs. It may be helical or generally cylindrical or tubular in shape and it may be a diamond-patterned expansile stent. The device is simple to insert into the patient and easy to retrieve from the patient and maintains positional stability, i.e., the device does not migrate either caudally or distally following implantation. The device does not represent a hazard to its recipient that exceeds its cumulative benefit, either in its anticipated placement or in those that might occur by accident. It does not obstruct the normal passage of chyme, from the pylorus for example, or the passage of bile and enzymes from the Ampulla of Vater. Rather it enables unrestricted chyme absorption within the gut small intestine and unrestricted chyme flow throughout the gut. The outward pressure exerted by the device itself evokes clinically meaningful responses, but not so great as to distort the normal architecture of the duodenum, or other gut segments in which it may be deployed, or to create erosions or discomfort. The device will advantageously not otherwise interfere with chemosensory, digestive or absorptive functions of the duodenum, or other gut segments in which it may be deployed. The result of the gut distension is to evoke not only perceptible satiogenic i.e., “fullness” effects by itself, but also therapeutically useful autonomic (automatically produced by internal stimuli) and humoral (hormonal, especially endocrinal) responses associated with volume/flow disturbance signals, some of which may not be sensed.

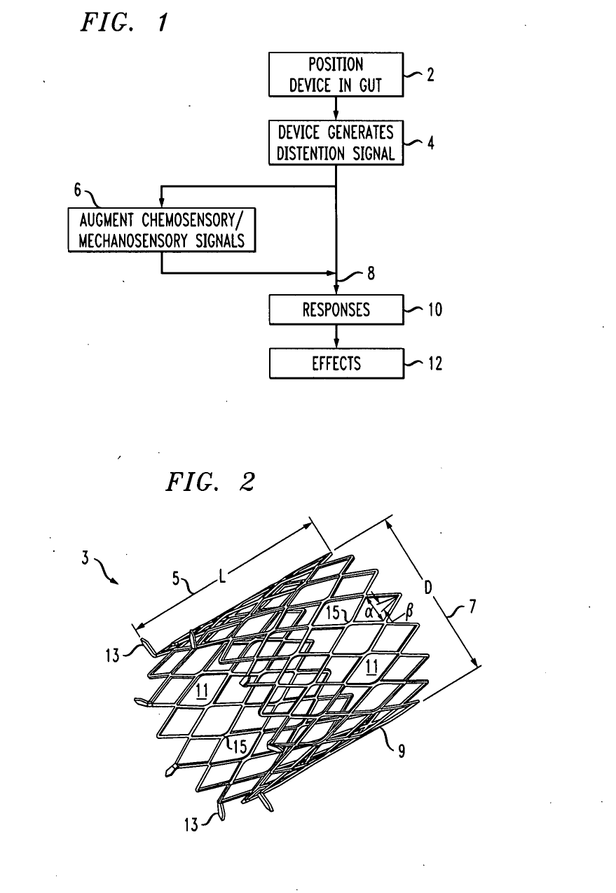

FIG. 1 is a flow chart showing aspects of the invention. At step (2), the device is positioned within the gut. The device may be implanted using various techniques. Exemplary sites at which the device may be implanted include the duodenum, i.e. proximal-, mid- and/or distal-duodenum, the jejunum, the ileum and the rectum. According to various exemplary embodiments, a device may be positioned at more than one or all of the aforementioned locations. Contiguous and non-contiguous combinations of the above placement locations can be used, i.e., a plurality of devices may be deployed simultaneously. At step (4), the device generates distension signals. The generation may be automatic or it may be remotely aided by an external stimulus. The distension signals induced by the device may be transmitted via neural pathways, humoral pathways, or both. The signals may, at step (6), augment chemosensory and/or other mechanosensory signals or their therapeutic mimics to create or amplify a therapeutic response. The chemosensory and/or other mechanosensory signals may be artificially induced by a pharmaceutical or other therapy. The responses, step (10) evoked by signals (8) include satiety, the limiting of food intake, gastric emptying and deceleration in the rate of digestion. Effects (12) of these responses include a reduction in body weight, reduction of plasma glucose and other nutrients and a reduction in associated comorbidities.

The device implantation may advantageously be endoscopic but other techniques include laparoscopic or open surgical implantation, and remote implantation, for example, via fluoroscopic control. The device may be implanted for acute effect, or it may reside permanently within an individual. Embodiments of the device may incorporate a deployment/extension system to promote the intraluminal positioning of the device in its correct anatomical position. Any deployment system known in the art or later developed may be used. Insertion of self-expanding devices may be via a containing sleeve, and retrieval via a snare, hook or similar device, typically under endoscopic control. Many endoscopists are already skilled in implanting devices within the gastrointestinal tract, and already possess the tools necessary for the procedure. Few specialist tools are necessary. A dedicated retrieval system to explant the device may be used, i.e. the device may include elements that assist in its retrieval, including snares, deflators and other retrieval systems known in the art or later developed.

The device may advantageously include a fixation or stabilization system to stabilize and position it such that it is maintained in relation to associated anatomical structures, is not passed or regurgitated, and will not obstruct flows. Embodiments which promote stability of position may include conformity to gut profiles and shapes. One example is an expansile ring that sits within the duodenal cap in a manner analogous to that of the cervical diaphragm contraceptive device.

Other expansile and anchoring systems may be spring-loaded. Anchoring systems may include the use of prongs, barbs or other elements that penetrate the tissue surface, or otherwise augment frictive properties. Some embodiments of the device include adhesives either delivered separately, or incorporated into the device.

Exemplary sites of delivery include, without limitation, proximal-, mid-, and distal-duodenum, proximal-, mid-, and distal-jejunum, proximal-, mid-, and distal-ileum, rectum, and combinations thereof, contiguous and non-contiguous. An advantageous site is at the duodenum.

In some embodiments, the device may remain in situ for months or longer for certain applications. In such embodiments, the device has certain attributes. For example, the device is typically made from materials that will not be toxic or cause chemical irritation. That is, the device will not act upon the body in other than a therapeutic mode. In another aspect, the body will not act upon the device. The device is substantially impervious to bile and other physiologic fluids, and will not be affected by large changes in pH or other features of the fluids which it will contain/contact.

The materials used to form the device may include biocompatible “memory” metals, e.g. nitinol, stainless steel, titanium and other surgical metals. The materials used to form the device may advantageously be biodegradable, such as poly(lactic-co-glycolic acid) (PLGA), to obviate later retrieval. The biodegradable material may be chosen to last for a therapeutically useful period, i.e., the device may degrade after treatment of the patient is complete. Biologically inert materials can be used in other exemplary embodiments, including Teflon and other plastics.

According to another aspect, the device is externally traceable. Materials utilized in the device may, for example, be radio-opaque, including having distinctive markers at certain places on the device, to assist with placement, assessment of position and function, and with other aspects of clinical management. Other contrasting techniques such as enhanced imaging assisted placement techniques may also be used to aid in placement. These techniques include X-ray contrasting, ultrasound contrasting, MRI contrasting and Remission. The device may include markers detectable using the associated imaging technique to aid in placement.

According to various exemplary embodiments, the device can be in the form of an annular wave, such as described in US Patent Publication 2005/0125020A1, in the form of a diamond mesh, or in the form of a self-expanding wire mesh, as in U.S. Pat. No. 6,675,809, each of which are incorporated herein by reference as if set forth in their entireties. In one embodiment, expansion of the distender device may be externally controllable, for example by fluid inflation, heating of memory metal, remote adjustment, or automatically adjusting. In another exemplary embodiment, the device may take the shape of various coiled springs, including those that expand upon release from an obturator. Diamond-pattern expansile stents, or other expanding surfaces may be used in other exemplary embodiments while inflatable struts and other fluid- or gas-containing configurations may alternatively or additionally be used. In some exemplary embodiments, the outward pressure exerted on the gut wall may be provided by the biocompatible “memory” metals such as nitinol. The implanted device may be a spring that provides a radially outward spring force that is sufficient to anchor the device in position and evoke therapeutically useful signals that are at least one of neural signals and humoral signals and directly or indirectly bring about the sensation of satiety. The outward pressure supplied by the device causes the expansion or distension of the portion of the gut anatomy in which it is deployed.

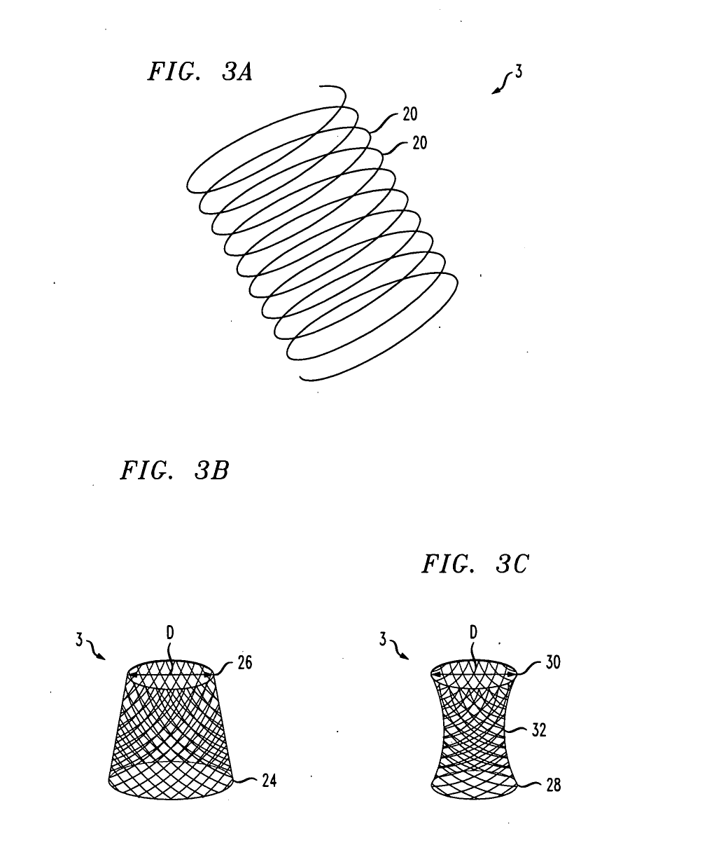

An exemplary gastrointestinal implant device of the invention is illustrated in FIG. 2. Duodenal distender device 3 includes length 5 and diameter 7 and may advantageously be implanted in the patient's small intestine or other gut locations. Length 5 may be on the order of 8-40 cm in one exemplary embodiment, 2-8 cm in another exemplary embodiments or it may exceed 40 cm. Length 5 may vary in other exemplary embodiments and may advantageously be tailored to a particular patient or for a particular response, i.e. it may be tailored to provide desired structural and/or physical properties, including comformation to general gut contours, or to specific contours of a specific individual.

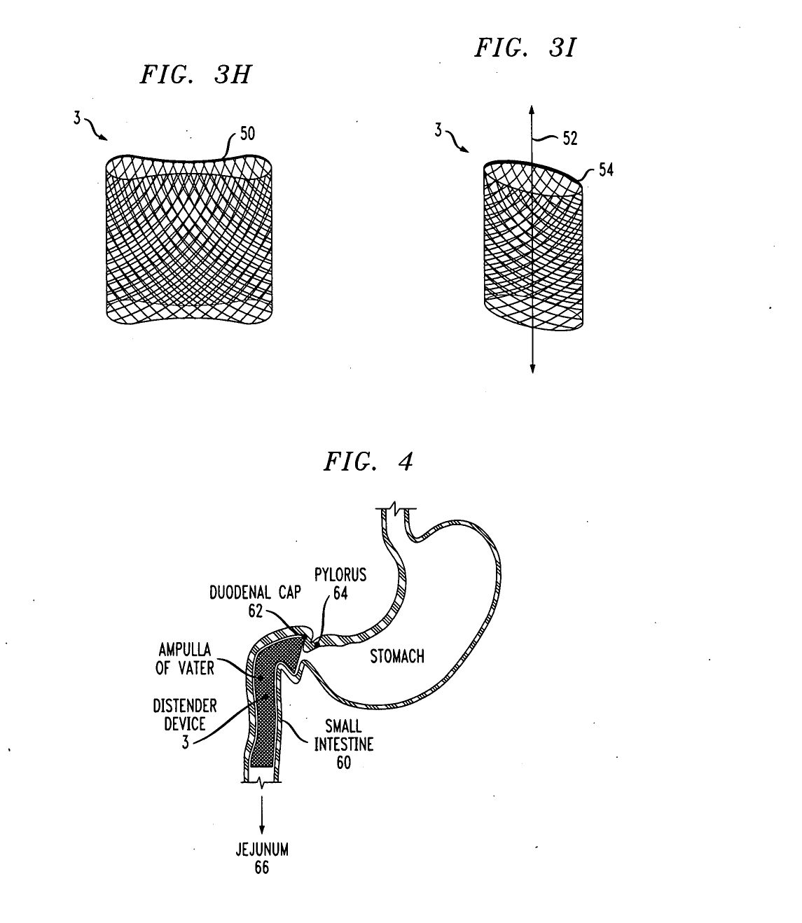

In the illustrated embodiment, device 3 is formed of a wire mesh pattern 9 which occupies only a small percentage of the overall device area. Only a small portion of the overall area occupied by the device—defined by L(x)πD—is actually occupied by material 15 with the remainder consisting of openings 11. In one exemplary embodiment, the actual material 15 may make up only about 25% or less of the overall area occupied by the device, i.e., the device may have a material density of less than 25%. In other exemplary embodiments, material 15 may make up only about 10% or less of the overall area occupied by the device. A plurality of peripheral openings 11 make up the sides of device 3 such that when device 3 is inserted into a patient's duodenum or small intestine, absorption between the chyme flowing through the device and the walls of the gut upon which device 3 provides an outward (expansile or spring) force, is largely uninterrupted. Moreover, the low material density of device 3 provides flexibility to the device which may bend and be contoured to be properly positioned in a patient as may be seen in FIG. 4.

Fixation members 13 are simple barbs in the illustrated embodiment used to secure device 3 in place within the patient. In other exemplary embodiments, other fixation devices or anchors may be used. In one exemplary embodiment, diameter 7 may be greater than length 5. It should be understood that the diamond-pattern expansile stent embodiment illustrated in FIG. 2 is intended to be exemplary only. Material 15 used to form device 5, i.e. mesh pattern 9, are as described supra and, infra. Materials 15 used to form device 3 provided herein are chosen to provide desired structural and/or physical properties including but not limited to, structural integrity. In another aspect, the external surface of device 3 is selected to minimize adherence to tissue, and to minimize retention of bacteria.

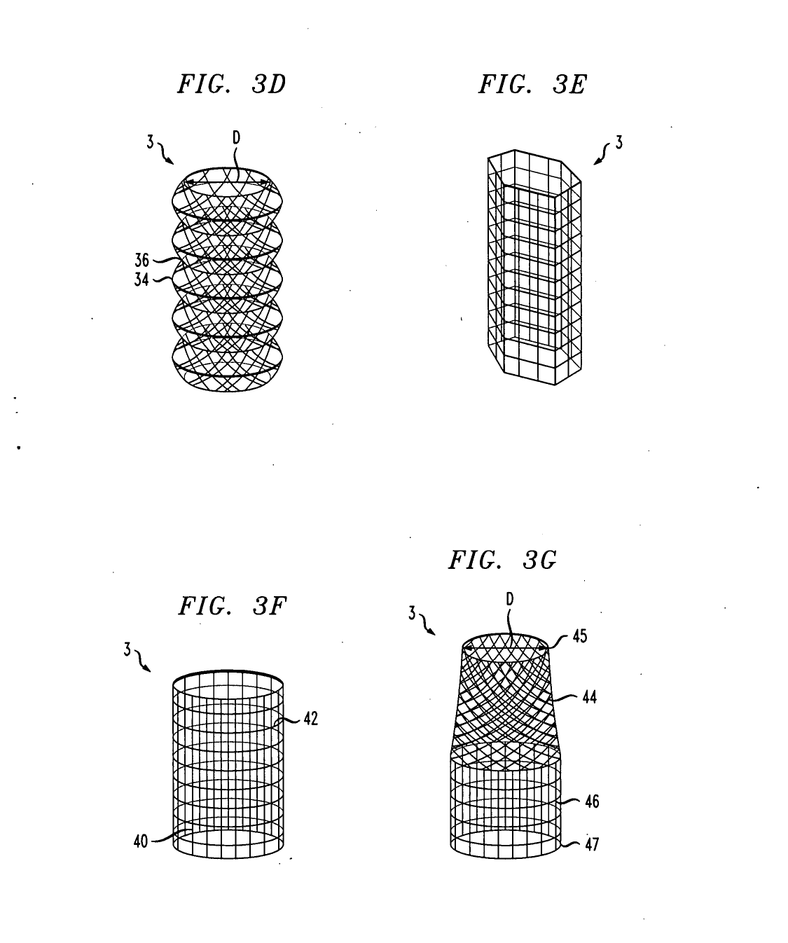

FIGS. 3A-3I show other exemplary embodiments of the distender device of the present invention. In FIG. 3A, the distender device 3 is in the form of a coiled spring and the coiled spring may be made of the previously discussed materials. The density of the coiled spring, i.e., the distance between adjacent spring coils 20, may vary in various exemplary embodiments and it may vary in any one embodiment, i.e., the spacing of spring coils 20 may be irregular.

FIG. 3B illustrates an exemplary embodiment in which the diameter D is greater at first end 24 than at second end 26, i.e., distender device 3, while generally tubular, is somewhat conical. Now turning to FIG. 3C, exemplary distender device 3 includes a gradually varying diameter. The diameter D at the ends 28, 30, is greater than the diameter at location 32 providing an hourglass-type configuration. In this manner, the contoured distender device may be customized for insertion into various locations. FIG. 3D shows an exemplary distender device 3 with a regularly-varying diameter D. It can be seen that the diameter at locations 34 is greater than the diameter at locations 36. Whereas the illustrated embodiment of FIG. 3D shows a diameter that varies regularly along the length of the distender device 3, in other exemplary embodiments, the diameter variation may be irregular.

FIG. 3E shows a generally tubular, hexagonally-shaped exemplary embodiment of another distender device 3. FIG. 3F illustrates yet another exemplary embodiment of distender device 3 formed by longitudinally extending ribs 40 which join together a plurality of rings 42. FIG. 3G shows another distender device and illustrates an embodiment in which the structural pattern of the distender device changes throughout the device. Portion 44 of distender device 3 consists of a diamond-shaped mesh pattern formed by diagonally crisscrossing lines whereas portion 46 consists of a mesh formed by rectangular units. FIG. 3D also shows the distender device 3 having a diameter that differs at respective ends 45, 47. FIG. 3H illustrates another exemplary embodiment of a non-circular, cylindrically-shaped distender device 3. Alternatively stated, two-dimensional curve 50 is not a circle and forms a non-circular cylinder as the two-dimensional curve 50 is projected along an axis intersecting the plane of two-dimensional curve 50 to form a cylinder. FIG. 31 shows another exemplary embodiment of a generally cylindrical shape for an exemplary distender device 3. In particular, FIG. 31 is not a right cylinder, i.e., the axis (52) of the cylinder is not perpendicular to the plane of the two-dimensional curve 52.

In other exemplary embodiments, the distender device 3 may take on still other generally cylindrical or tubular shapes and may have a cross-section that is constant or one that varies in size along the length of the device and/or a cross-section that is elliptical or has other non-circular configurations.

FIG. 4 shows an exemplary distender device 3 deployed in the duodenal portion of the small intestine 60 and extending to duodenal cap 62 adjacent to pylorus 64. Distender device 3 is bent and conforms to the part of the small intestine between the stomach and jejunum 66 but this is intended to be exemplary only and in other exemplary embodiments, distender device 3 may take on other configurations and may be relatively shorter or longer than as illustrated in the exemplary embodiment of FIG. 4, and may occupy different lengths of the patient's duodenum/small intestine. In other embodiments, not shown, the distender device may be deployed in the jejunum, ileum or rectum.

The device and method of treatment according to the invention is believed to have numerous advantages over previous devices and methods. Compared to the stomach, motility and churning is less in the small bowel, resulting in a reduced chance of displacement or of device breakage relative to gastric distenders.

In addition to the materials and configurations illustrated and described herein, embodiments of the device also include material and engineering improvements made in the future. The applied force provided by the device may be passive such as a spring-based force, or active, utilizing motive energy, which may be supplied externally or tapped from within the body. The applied outward force may be reactive, e.g. a component of an automated feedback loop. Once implanted, the device may be self expanding, e.g. elastic, or formed of the aforementioned “memory materials” that cause an expansion upon a temperature change. Other conventional external events or signals including motorized activation may cause the device to expand to fit snugly against the small intestinal wall. The device may simply be spring loaded and chosen to automatically and continually exert a radially outward spring force necessary to distend the portion of the gut in which it is deployed. The amount of expansion and/or the degree of applied outward force may be automatically adjusted or set, or it may be selectively controlled externally using any of various commercially available devices that remotely and externally control the degree of expansion of the device or the amount of force applied radially outward. The expansile or other outward force may be constant or intermittent, and need not be the same at all parts of the device, i.e., the device need not be isobaric. For example, a device formed of a spring may be a spring that exerts different amounts of outward force at different longitudinal locations. The force may be modulated by inputs that include those from the patient or other operator, conferring the capacity to tune the device during operation. In various exemplary embodiments, the device may distend the anatomy directly or indirectly.

An important aspect of the invention is that the force supplied by the device, is itself sufficient to produce the aforedescribed distension signals that bring about the effects described herein. The device does not require any other aspects or effects (e.g. electrodes or electrical or other signals) to treat obesity.

Without being bound to the following mechanism, when deployed in the small intestine, the effect of the device is to distend the small intestine and generate signals that would ordinarily indicate that filling of the small bowel was too rapid. The effect of such signals will be either alone, or in combination with other meal-derived or pharmaceutically induced signals, to evoke corrective responses that collectively limit nutrient influx into the small bowel. Such responses include satiety, limiting food intake, gastric emptying, and deceleration in rate of digestion. The orchestrated effect of such a combination of signals will be a reduction in body weight, reduction of plasma glucose and other nutrients, and a reduction in associated comorbidities.

The nature of the induced distension signals may be neural, within the enteric and autonomic nervous systems, or may be humoral, causing the secretion of locally-acting and systemically-acting signals (such as peptide hormones), or both. The systematically-acting signals propagate through the body and advantageously to the brain.

Distension signals typically, but not necessarily, act in association with other signals, such as the enteroendocrine secretions that typically result from detection of both nutrient and non-nutrient chemosensory stimuli in the gut lumen. The enteroendocrine secretions may be autonomic (endogenous) digestive responses of the body arising in response to meals or they may be caused, assisted or mimicked pharmaceutically. The distension device of the present invention can thus act synergistically with therapies that aim to promote or emulate chemosensory signals. More particularly, the distending device may augment one or more chemosensory or other mechanosensory signals. The chemosensory signals evoke corrective metabolic responses that produce weight loss in the patient, the corrective metabolic responses including satiety, limitation of food intake, slowing of gastric emptying and a deceleration in the rate of digestion. Such therapies include pharmaceuticals such as gut peptides or their mimics, of potentiators of those signals such as inhibitors of dipeptidyl peptidase 4 and other peptide degrading enzymes, secretagogues of peptides, and devices that aim to evoke chemosensory signals.

Examples of enteroendocrine secretions include but are not limited to the following. At the stomach, gastrin is secreted in response to calcium, amino acids and fermented glucose. Gastric inhibitory polypeptide (GIP), secretin and cholecystokinin (CCK) are secreted in response to fat; CCK and GIP in response to duodenal glucose; GIP and CCK in response to certain amino acids. Responses to protein meals depend upon their breakdown to amino acids. Neurotensin and glucagon-like peptide-1 (GLP)-1 are secreted in response to fat and carbohydrate in the ileum. Specific mechanisms sensing these nutrient signals are generally not characterized, but can include receptors on apical microvilli of endocrine cells or indirect sensing via the intrinsic nervous system and/or accessory cells.

A long-recognized example of nutrient sensation in the gut is exemplified in its ability to respond to fat. For example, long chain fats (C12 or greater), drive CCK stimulation within minutes of application. GLP-1 is also secreted in response to fat, but apparently depends on at least partial digestion, since responses are blunted when a lipase inhibitor is added as discussed in Pilichiewicz, A., O'Donovan, D., Feinle, C., Lei, Y., Wishart, J. M., Bryant, L., Meyer, J. H., Horowitz, M. and Jones, K. L. (2003), Effect of lipase inhibition on gastric emptying of, and the glycemic and incretin responses to, an oil/aqueous drink in type 2 diabetes mellitus, J Clin Endocrinol Metab 88, 3829-3834. Understanding of chemosensory mechanisms within the gut is presently rudimentary, but can include the same receptors responsible for taste at the tongue, Wu, S. V., Rozengurt, N., Yang, M., Young, S. H., Sinnett-Smith, J. and Rozengurt, E. (2002), Expression of bitter taste receptors of the T2R family in the gastrointestinal tract and enteroendocrine STC-1 cells, Proc Natl Acad Sci USA 99, 2392-2397.

Gut peptides that are secreted in response to intraluminal meal-related stimuli represent a further enteroendocrine secretion and are shown in the following table. Several, for example, CCK, GLP-1, PYY, oxyntomodulin, neurotensin, inhibit feeding, and through anorectic and/or other mechanisms, can induce weight loss. The present invention provides a distension signal that amplifies such enteroendocrine gut peptide secretions including those listed below.

| | | Cells |
|---|---|---|
| Peptide | Luminal Secretagogue | of Origin |
| Gastrin | Esp. aromatic amino acids and amines | G cells |
| Somatostatin | Intragastric acid | D cells |
| Secretin | Intraduodenal acid | S cells |
| CCK | Fats, proteins | I cells |
| GIP | Carbohydrates, triglycerides | K cells |
| Motilin | Poss. duodenal alkaline | M cells |
| GLP-1, -2 | Carbohydrates (incl. non-metabolized) | L cells |
| Pancreatic | Vagal, intraluminal amino acids, glucose, | PP cells |
| Polypeptide (PP) | fat | |
| Peptide YY | Intraluminal fat, protein | L cells |
| Oxyntomodulin | Intraluminal fat | L cells |
| Neurotensin | Jejunal fat | N cells |

The site of release of such gut peptides is variable. Tissue concentrations of PYY, for example, are known to increase with progression down the gut. The site of release is not necessarily predicted by tissue content. For example, even though tissue content continuously increases with progression along the gut, most release of GLP-1 is considered to come from the terminal ileum, by which segment most nutrient is absorbed. Presence of gut peptides beyond that level probably represents a “fail-safe” in that, with progression down the gut, increasingly vigorous secretion occurs in the decreasingly probable event that nutrient or other secretagogues reach there.

Examples of therapeutic peptides conveying a nutrient sense include amylin, CCK, GRP, GLP-1, oxyntomodullin, insulin, PYY, leptin, neurotensin, urocortins, neuromedins and agonists thereto. Distension signals, as generated by the current device can synergize with such peptides.

Distension signals, as generated by the current device can likewise synergize with or otherwise augment chemosensory signals other than peptides. The chemosensory signals may be peptide signals artificially induced by pharmaceuticals. Such chemosensory signals can be nutrient signals, or mimics of nutrient signals, as are evoked by stimulation of the many fuel/nutrient-sensing receptors within the gut, including taste receptors, glucose sensors, fatty-acid receptors and the like.

Distension signals, as generated by the current device can similarly include non-peptide non-nutrient signals, such as those generated by Toll receptors, part of the immune defense that maintains a barrier between gut organisms and the body interior. A further example of non-peptide non-nutrient signals are bile salts, the presence of which in the lower small bowel evokes anorectic and gastric inhibitory responses to allow better bile salt recuperation into the recirculating bile salt pool.

Examples of devices that promote the latter chemosensory signals include the biliary shunt described in U.S. Provisional Patent Application 60/729,770, and the enzyme sleeve described in U.S. Provisional Patent 2004/0249362 A1.

The invention provides a method for treating obesity using the aforedescribed device. The method includes inserting the device, capable of imparting an outward force, in a human or other patient. The outward force may be an expansile force or spring force and it distends, i.e., causes the expansion of, the gut section in which it is deployed. The device may be inserted into the patient's small intestine, including the duodenum, jejunum and ileum or rectum. The method also includes implanting the distension device according to the techniques described above. The method further includes advantageously positioning the device in an intended location using the aforementioned aspects of the invention in conjunction with conventional techniques. The method includes the device generating a distension signal in the small intestine by expanding the small intestine. The applied force may be an automatic force, e.g. spring-loaded expansion of the inserted device and it also may be effectuated by an outside stimulus. The method also includes causing the device to produce a distension signal and the distension signal amplifying one or more chemosensory signals within the patient. The method includes the distension signal, in conjunction with the amplified or augmented chemosensory signals, evoking responses in the patient that produces weight loss in the patient. The method further includes optionally removing the device when appropriate.

The following list provides various aspects of the invention in tabular form and is supplemental to the above detailed description and is illustrative and exemplary of the invention and not limiting. The following tabular presentation of “aspects of the invention” is therefore to be read in conjunction with the previously described detailed description.

Aspects of the Invention:

Device used in treatment or prevention of at least one of
diabetes
impaired glucose tolerance
glucose metabolic disorders
insulin resistance
obesity

The use of an intestinal or rectal distender combined with
a peptidic satiogenic signal
   amylin, calcitonin (including teleost), intermedin, CRSP or CGRP agonist
   CCK or agonist
   GLP-1 or exendin agonist
   PYY or agonist
   oxyntomodullin, glucagon or agonist
   leptin, CNTF or agonists
   melanocortin agonists or agonists of other POMC or agouti gene products
   neurotensin, urocortin, neuromedin, endothelin agonists
a therapy that promotes endogenous satiety signals
   inhibitors of peptidases
      dipeptidyl peptidase
      neutral endopeptidase
a thermogenic (heat wasting) therapy
an inhibitor of nutrient assimilation, including
   inhibitors of digestive function
   inhibitors of digestive secretions
      gastric acid
      enzyme secretion
      bile secretion
   inhibitors of digestion, enzyme inhibitors
      lipase
      glucosidase
   inhibitors of absorption, inhibitors of nutrient transporters
      glucose cotransport inhibitors
      amino acid transport inhibitors
      lipid transporter inhibitors
      bile salt transport inhibitors
      metformin and other biguanides

In one further exemplary embodiment of the invention, a single device may be constructed that accommodates both the present invention and the invention described in U.S. Provisional Patent Application No. 60/729,770 entitled Biliary/Pancreatic Shunt Device and Method for Treatment of Metabolic and Other Diseases by the Applicant, the contents of which are incorporated by reference as if set forth in its entirety. For example, a duodenal distender device may also act to position a collection manifold over the Ampulla of Vater to capture pancreaticobiliary secretions. Bile-containing secretions are then directed via a conduit of some description to distal segments of the gut.

Aspects of the invention also include the use of the distension device in combination with other devices, treatments and pharmaceuticals to effectuate two or more actions, including but not limited to the following: intestinal distension, especially duodenal distension; pancreaticobiliary shunting, i.e. the delivery of bile to a more distal intestinal site than normally occurs, as described in U.S. Provisional Patent Application 60/729,770; enzyme shunting, i.e. the delivery of digestive enzymes to a more distal intestinal site than normally occurs, as described in U.S. Patent App 2004/0249362A1; nutrient shunting, i.e. the delivery of nutrient to a more distal intestinal site than normally occurs, as described in WO 04/049982A2, U.S. Patent Application publications 2004/0107004, 2005/0080395 and U.S. Patent App. 2005/0125020A1; and electrically generated satiety/distension signalling as evoked, for example by vagal stimulation, as described in U.S. Pat. No. 6,535,764 directed to an intraluminal gastric stimulator

The written description portion of this patent application includes all claims. Furthermore, all claims, including all original claims as well as all claims from any and all priority documents, are hereby incorporated by reference in their entirety into the written description portion of this specification, and Applicant reserves the right to physically incorporate into the written description or any other portion of the application, any and all such claims. Thus, for example, under no circumstances may the patent application be interpreted as allegedly not providing a written description for a claim on the assertion that the precise wording of the claim is not set forth in haec verba in the written description portion of the patent application.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of exemplary embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes any generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun.

Other embodiments are within the following claims. Any patent issuing from this application may not be interpreted to be limited to the specific examples or embodiments or methods specifically and/or expressly disclosed herein. Under no circumstances may such patent be interpreted to be limited by any statement made by any Examiner or any other official or employee of the Patent and Trademark Office unless such statement is specifically and without qualification or reservation expressly adopted in a responsive writing by Applicant.

What is claimed is:

1. A method for treating obesity and/or lowering of weight or adiposity in a patient comprising:
inserting at least one device capable of exerting an outward force in said patient's small intestine, said device allowing substantially unrestricted chyme absorption within said small intestine and substantially unrestricted chyme flow throughout said small intestine;
generating a small intestinal distension signal by exertion of said outward force by said device only; and
said outward force causing said small intestinal distension signal to thereby evoke weight loss or satiogenic effects by itself.

2. The method as in claim 1, wherein said device exerts said outward force by expansion.

3. The method as in claim 2, wherein said expansion is remotely and externally controllable.

4. The method as in claim 1, wherein said outward force comprises a spring force.

5. The method as in claim 1, wherein said outward force comprises a motive force and an amount of said outward force is remotely and externally controllable.

6. The method as in claim 1, wherein said inserting comprises inserting in said patient's duodenum adjacent said patient's pylorus.

7. The method as in claim 1, wherein said small intestinal distension signal augments one or more chemosensory signals within said patient and wherein said chemosensory signals are generated responsive to a meal.

8. The method as in claim 1, wherein said small intestinal distension signal amplifies one or more chemosensory or mechanosensory signals within said patient and wherein said one or more chemosensory or mechanosensory signals evoke corrective metabolic responses that produce weight loss in said patient.

9. The method as in claim 8, wherein said corrective metabolic responses that produce weight loss in said patient include satiety, limitation of food intake, slowing of gastric emptying and a deceleration in rate of digestion.

10. The method as in claim 1, wherein said weight loss or satiogenic effects include satiety, limitation of food intake, slowing of gastric emptying and a deceleration in rate of digestion.

11. The method as in claim 1, wherein said exertion of said outward force induces therapeutically useful signals that are at least one of neural signals and humoral signals that include one of peptides and non-peptides.

12. The method as in claim 11, wherein said peptides comprise at least one of CCK, GLP-1, GLP-2, PYY, gastrin, somatostatin, secretin, GIP, motilin, pancreatic polypeptide, peptide YY, oxyntomodulin, neuromedin and neurotensin.

13. The method as in claim 1, further comprising said method treating other cardiovascular risk factors including at least one of dysglycemia, dyslipidemia and hypertension.

14. The method as in claim 1, further comprising said device automatically adjusting an amount of said outward force.

15. The method as in claim 1, wherein said small intestinal distension signal comprises at least a humoral signal that would normally follow ingestion of a meal.

16. The method as in claim 1, wherein said small intestinal distension signal comprises enteroendocrine secretions.

17. The method as in claim 1, further comprising introducing a pharmaceutical therapy to said patient and wherein said small intestinal distension signal amplifies one or more chemosensory signals within said patient, said chemosensory signals generated in response to said pharmaceutical therapy.

18. The method as in claim 1, wherein said patient is a human and said device exerts said outward force by self-expanding.

19. The method as in claim 1, wherein said device is formed of a biodegradable material and further comprising said device degrading after a treatment of said patient.

20. The method as in claim 1, wherein said patient is a human and said small intestinal distension signal induces at least one of a neural signal and a humoral signal within said patient.

21. The method as in claim 1, wherein said inserting comprises endoscopically inserting and further comprising using an enhanced imaging placement technique comprising one of X-ray contrasting, ultrasound contrasting, MRI contrasting and γ-emission to position said device.

22. The method as in claim 21, wherein said using said enhanced imaging placement technique includes utilizing externally detectible imaging markers on said device.

23. A method for treating obesity a human comprising:
inserting a device capable of imparting an expansile force in a human's small intestine;
generating a small intestinal distension signal by expanding said device to provide a mechanical force sufficient to induce therapeutically useful signals that are at least one of neural signals and humoral signals that evoke satiogenic or weight loss effects by themselves while allowing for unrestricted chyme absorption within said small intestinal, unrestricted chyme flow through said small intestinal and unmodified further digestive functions; and
said small intestinal distension signal amplifying chemosensory signals within said human,
wherein said chemosensory signals are one of generated responsive to a meal and generated responsive to a pharmaceutical therapy.

24. A method for treating obesity and/or lowering of weight or adiposity in a patient comprising:
inserting at least one device capable of exerting an outward force in said patient's rectum;
generating a rectal distension signal by exertion of said outward force by said device only;
said outward force causing said rectal distension signal to thereby evoke weight loss or satiogenic effects by itself; and
said rectal distension signal amplifying one or more chemosensory signals within said patient.

25. An insertable weight loss apparatus for a patient comprising a gastrointestinal implant consisting of a discrete tubular-shaped device adapted for deployment in said patient's small intestine and for exerting physical pressure radially outward sufficient to evoke a therapeutically useful distension signal of satiety while allowing substantially unrestricted chyme flow through said small intestine and substantially unrestricted chyme absorption in said small intestine.

26. The insertable weight loss apparatus as in claim 25, wherein said device comprises a helical shape.

27. The insertable weight loss apparatus as in claim 25, wherein said device comprises a cylindrical shape.

28. The insertable weight loss apparatus as in claim 27, wherein said device is a cylinder other than a right cylinder.

29. The insertable weight loss apparatus as in claim 25, wherein said device comprises an annular wave radial spring.

30. The insertable weight loss apparatus as in claim 25, wherein said device includes a diameter that varies in a longitudinal direction.

31. The insertable weight loss apparatus as in claim 30, wherein said device comprises a conical shape.

32. The insertable weight loss apparatus as in claim 30, wherein said device includes a diameter that varies in regularly repeating manner in the longitudinal direction.

33. The insertable weight loss apparatus as in claim 25, wherein said device includes a non-circular cross section.

34. The insertable weight loss apparatus as in claim 25, wherein said device comprises a mesh with a material density of less than 25%.

35. The insertable weight loss apparatus as in claim 25, wherein said physical pressure varies along a longitudinal direction of said device.

36. The insertable weight loss apparatus as in claim 25, wherein said device is formed of a biodegradable material that lasts until treatment of said patient is complete.

37. The insertable weight loss apparatus as in claim 25, wherein said device is formed of shape memory material.

38. The insertable weight loss apparatus as in claim 25, wherein said device is an expansile device.

39. The insertable weight loss apparatus as in claim 25, wherein said device has a length less than 40 centimeters.

40. The insertable weight loss apparatus as in claim 25, wherein said therapeutically useful distension signal amplifies one or more chemosensory signals.

41. The insertable weight loss apparatus as in claim 25, wherein said apparatus further consists of a fixation member that stabilizes said device in place.

42. The insertable weight loss apparatus as in claim 25, wherein said device is formed of an elastic material.

43. The insertable weight loss apparatus as in claim 25, wherein an amount of said physical pressure is externally controllable.

44. The insertable weight loss apparatus as in claim 25, wherein said physical pressure is provided by a motive force.

45. The insertable weight loss apparatus as in claim 25, wherein said device is formed of radio-opaque materials and includes markers that are detectable using imaging techniques when said device is inside said patient.
